(19) United States
(12) Patent Application Publication
(10) Pub. No.: US 2013/0001536 A1
(43) Pub. Date: Jan. 3, 2013

(54) NOVEL BENZOXAZOLYL CARBAZOLE COMPOUND AND ORGANIC LIGHT-EMITTING DEVICE INCLUDING THE SAME

(75) Inventors: Takeshi Sekiguchi, Tokyo (JP); Koichi Suzuki, Yokohama-shi (JP); Hajime Muta, Zama-shi (JP); Ryuji Ishii, Yokohama-shi (JP)

(73) Assignee: CANON KABUSHIKI KAISHA, Tokyo (JP)

(21) Appl. No.: 13/583,705

(22) PCT Filed: Mar. 14, 2011
(86) PCT No.: PCT/JP2011/056672

§ 371 Date: Sep. 10, 2012

(30) Foreign Application Priority Data
Mar. 29, 2010 (JP) 2010-075663

(51) Int. Cl. C07D 413/04; H01L 51/54; H01L 51/30

(52) U.S. Cl. 257/40; 548/222; 257/E51.026

(57) ABSTRACT

A novel organic compound which can be preferably used as a phosphorescent host material is provided. A benzoxazolyl carbazole compound represented by the general formula [1] shown in the specification is provided.

### TECHNICAL FIELD

The present invention relates to a benzoxazolyl carbazole compound which is a novel organic compound. In addition, the present invention relates to an organic light-emitting device including the above novel compound.

### BACKGROUND ART

An organic light-emitting device has the structure including a transparent substrate, a lower-side electrode, a laminate of organic compound layers, which contains at least one light-emitting layer, and an upper-side electrode provided in this order from a bottom side. The organic light-emitting device has attracted attention as one technique for a next generation full color display device having a high speed response, a high efficiency, and a flexible property, and hence, material technology development and device technology development have been energetically performed. In particular, an organic light-emitting device using electroluminescence is sometimes called an organic electroluminescence device or an organic EL device.

In recent years, in order to realize a higher efficiency, an organic light-emitting device using phosphorescence emission through triplet excitons has been actively developed. In a blue phosphorescent organic light-emitting device, a metal complex using iridium (Ir), such as FIrpic (bis(4,6-difluorophenylpyridinato-N,C2)picolinatoiridium), is used as a light-emitting material (guest material).

In recent years, in consideration of environmental preservation besides improvement in light-emitting efficiency, particularly, decrease in power consumption of display has been increasingly required, and development of decreasing a device operation voltage has also been carried out.

In a phosphorescent organic light-emitting device, since the performance of a host material is greatly reflected in device performance, host materials have been actively developed.

In order to simultaneously obtain a high efficiency and a decrease in device operation voltage of the phosphorescent organic light-emitting device, as compared to the guest material, the host material must have high lowest triplet energy and high hole and electron transportabilities. However, a host material which can be satisfactorily used in practice has not been developed.

PTL 1 has disclosed a device which uses a benzoxazole derivative substituted with an aryl group as an electron injection compound.

Since having high electron mobility, a benzoxazole structure can be suitably used as an electron transport layer; however, an aryl group has low hole mobility. That is, since having low hole mobility, the benzoxazole derivative substituted with an aryl group was not suitably used as a host material.

PTL 2 has disclosed a carbazole compound in which a phenyl group is bonded to nitrogen instead of hydrogen. Since having high lowest triplet energy and high hole mobility, the carbazole compound as described above has been widely used; however, the electron mobility thereof was not so high as compared to the hole mobility. Hence, when this carbazole compound was used for forming a device, the efficiency was low, and the operation voltage was high.

That is, in order to improve the efficiency and to decrease the device operation voltage, as a host material for a phosphorescent organic light-emitting device, a novel compound having high lowest triplet energy and high hole and electron transportabilities has been desired.

### CITATION LIST

### Patent Literature

PTL 1 Japanese Patent Laid-Open No. 2006-093673
PTL 2 Japanese Patent Laid-Open No. 2005-154412

### SUMMARY OF INVENTION

### Technical Problem

The present invention provides a novel organic compound. In addition, the present invention provides an organic light-emitting device which has a high light-emitting efficiency and which can be driven by a low voltage.

### Solution to Problem

A novel organic compound according to the present invention is a benzoxazolyl carbazole compound represented by the following general formula [1].

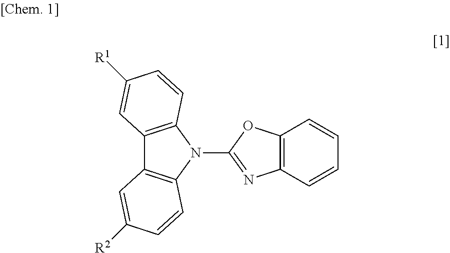

In the above formula, R¹ and R² independently indicate a non-substituted phenyl group or a phenyl group substituted with at least one alkyl group having 1 to 6 carbon atoms.

In addition, the organic light-emitting device according to the present invention has the following features. That is, in an organic light-emitting device which includes one or more organic layers disposed between a pair of electrodes which are arranged to face each other, at least one organic layer is a light-emitting layer containing the benzoxazolyl carbazole compound represented by the following general formula [1].

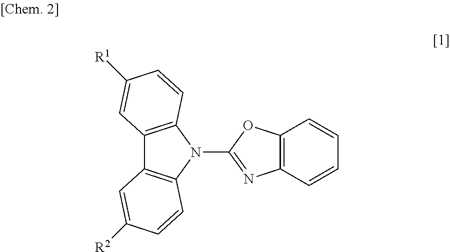

In the above formula, R¹ and R² independently indicate a non-substituted phenyl group or a phenyl group substituted with at least one alkyl group having 1 to 6 carbon atoms.

### Advantageous Effects of Invention

According to the present invention, a novel compound useful as a host material for a phosphorescent organic light-emitting device can be provided. In addition, an organic light-emitting device which has a high light-emitting efficiency and which can be driven by a low voltage can be provided.

### BRIEF DESCRIPTION OF DRAWING

FIG. 1 is a schematic cross-sectional view showing an organic light-emitting device and a switching device connected thereto.

### DESCRIPTION OF EMBODIMENT

A novel organic compound according to the present invention is an organic compound represented by the following general formula [1].

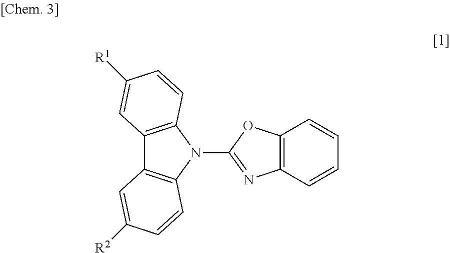

In the above formula, R¹ and R² independently indicate a non-substituted phenyl group or a phenyl group substituted with at least one alkyl group having 1 to 6 carbon atoms. Since R¹ and R² independently indicate a non-substituted phenyl group or a phenyl group substituted with at least one alkyl group having 1 to 6 carbon atoms, the lowest triplet energy can be maintained high. In particular, a tolyl group, a xylyl group, or a mesityl group is more preferable.

The molecular structure according to the present invention is formed by bonding benzoxazole to a carbazole compound. Accordingly, although the carbazole compound has a substituent, an energy state approximately equivalent to the triplet energy of carbazole can be maintained. In addition, benzoxazole is bonded to the nitrogen atom of the carbazole compound. Since the electron mobility of a benzoxazole group can be imparted to a carbazole group, compared to a carbazole compound substituted with an aryl group at the same position as described above, the electron mobility is improved. In addition, it has been known that the 3-position and the 6-position of carbazole are electrochemically active. Since the compound according to the present invention has the substituents R¹ and R² at the 3-position and the 6-position, electrochemical stability can be obtained, and when this compound is used for a light-emitting device, a stable device can be formed.

Hence, the benzoxazolyl carbazole compound according to the present invention can be used as a host material for blue phosphorescence having a short light-emitting peak wavelength.

Although particular examples of the benzoxazolyl carbazole compound according to the present invention are shown below, the present invention is not limited thereto.

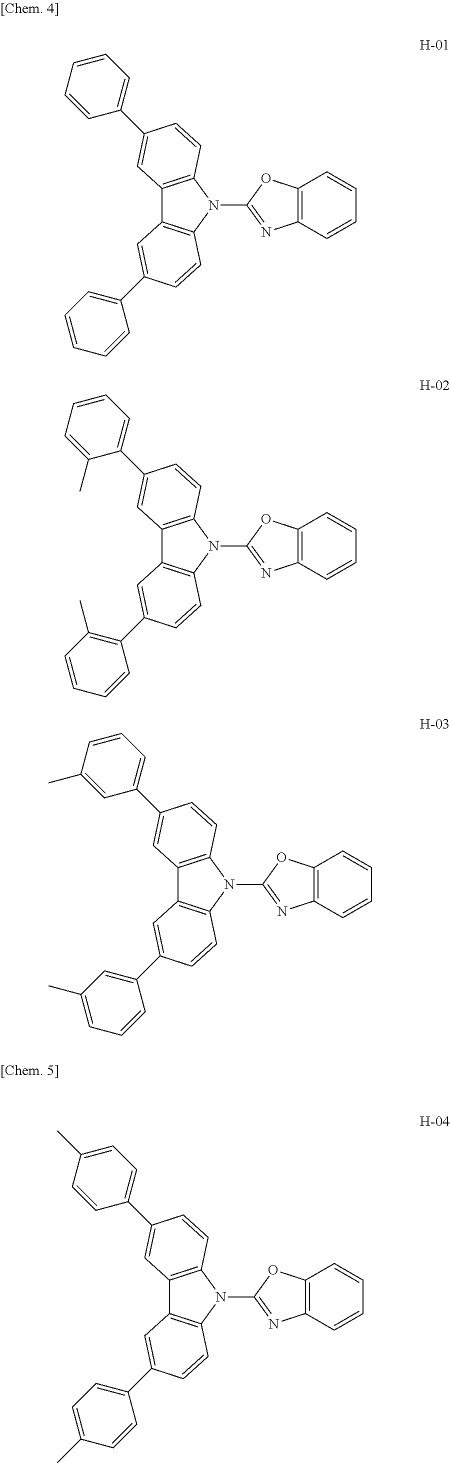

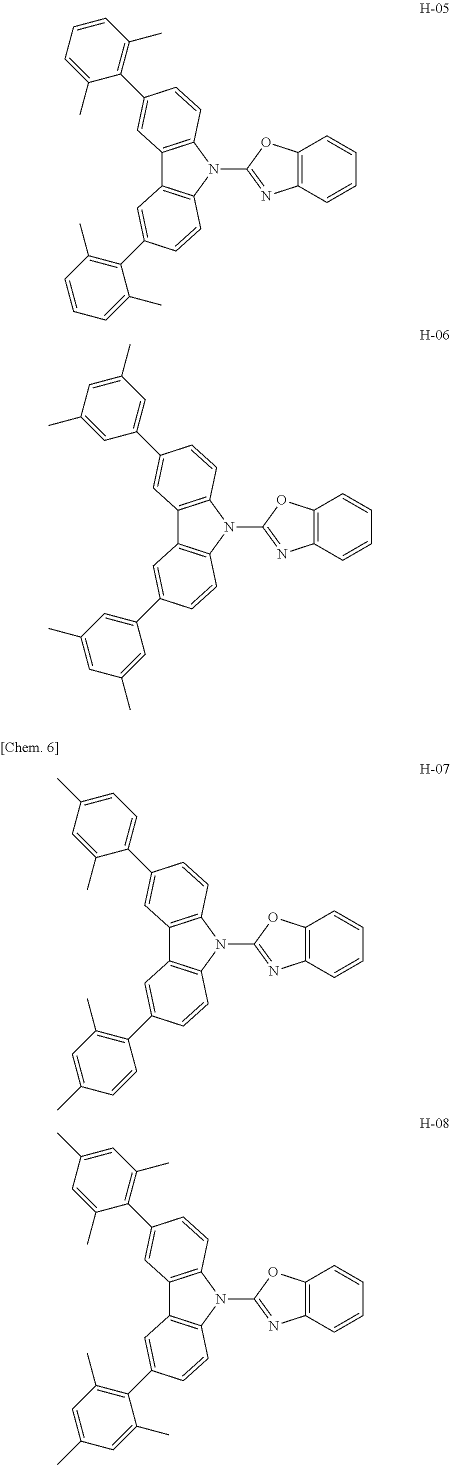

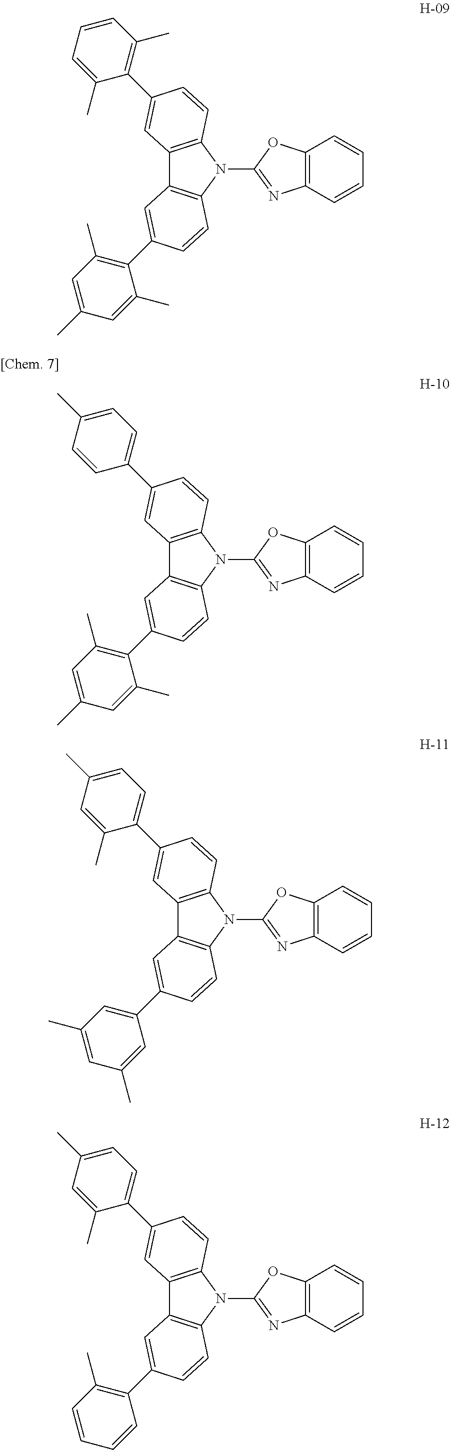

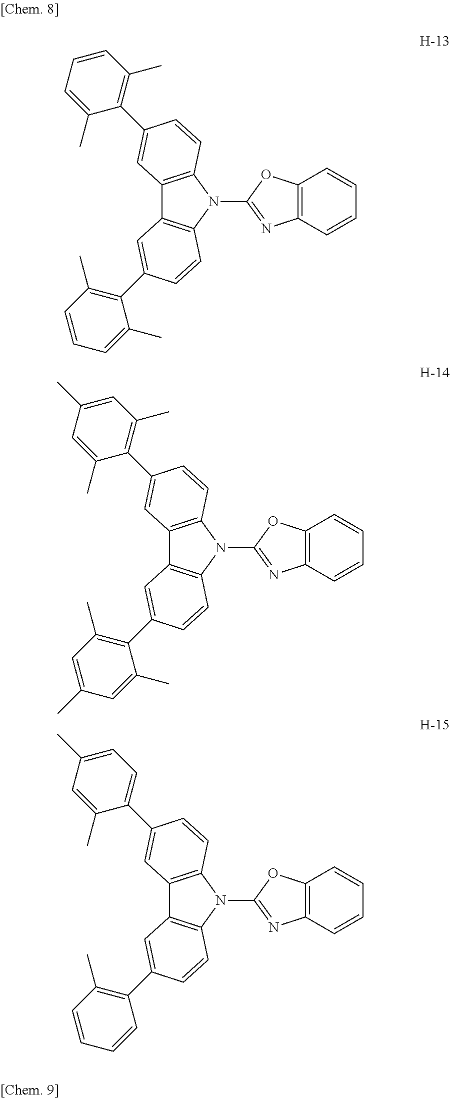

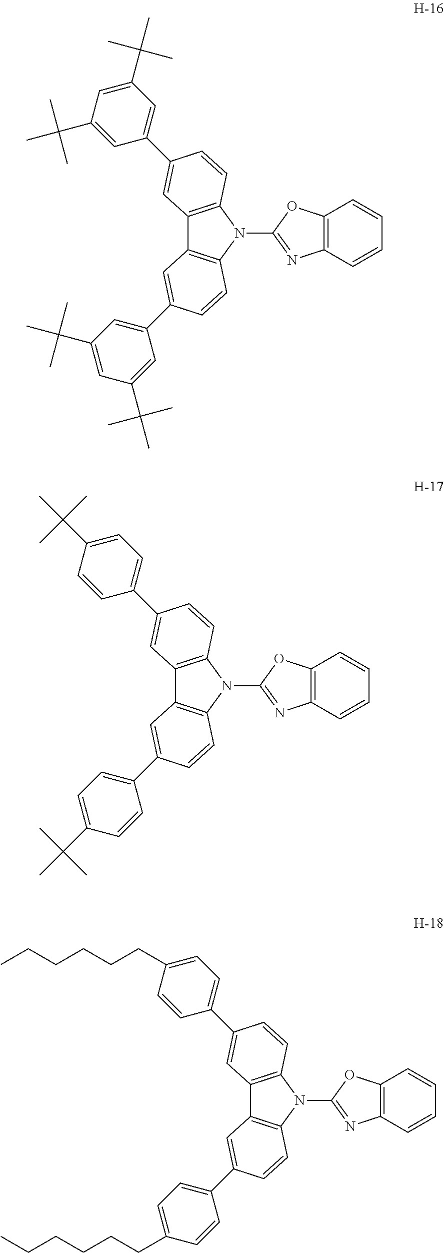 [SMILES: CC(C)(C)c1cc(-c2ccc3c(c2)c2cc(-c4cc(C(C)(C)C)cc(C(C)(C)C)c4)ccc2n3-c2nc3ccccc3o2)cc(C(C)(C)C)c1 ; CC(C)(C)c1ccc(-c2ccc3c(c2)c2cc(-c4ccc(C(C)(C)C)cc4)ccc2n3-c2nc3ccccc3o2)cc1 ; CCCCCCc1ccc(-c2ccc3c(c2)c2cc(-c4ccc(CCCCCC)cc4)ccc2n3-c2nc3ccccc3o2)cc1]

### Synthesis of Organic Compound

The organic compound according to the present invention can be synthesized by the following two-stage synthetic pathway which will be described in detail in Example 1.

### First Stage

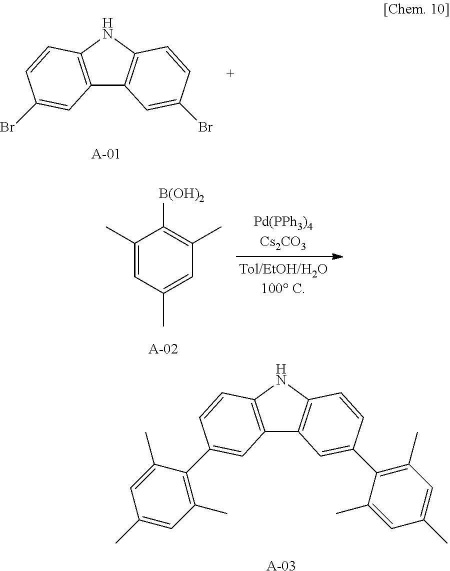

### Second Stage

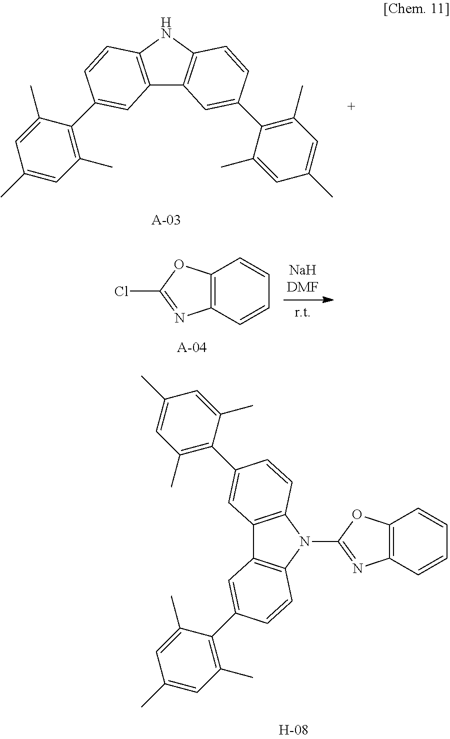

When a starting material A-02 on the first stage is changed, the benzoxazolyl carbazole compounds according to the present invention as shown above can be synthesized.

### Description of Organic Light-Emitting Device

Next, an organic light-emitting device according to this embodiment will be described.

The organic light-emitting device according to this embodiment is an organic light-emitting device which has one or more organic layers disposed between a pair of electrodes which are arranged to face each other, and at least one organic layer is a light-emitting layer containing the benzoxazolyl carbazole compound represented by the general formula [1].

As a light-emitting material used for the light-emitting layer, besides known light-emitting materials, a phosphorescent material is preferably contained in the light-emitting layer as a guest material. As the guest material, for example, a phosphorescent light-emitting Ir complex, such as FIrpic or FIr6, may be mentioned.

As a hole transport material used for a hole transport layer in the organic light-emitting device according to the present invention, for example, a triarylamine derivative, a phenylenediamine derivative, a triazole derivative, an oxadiazole derivative, an imidazole derivative, a pyrazoline derivative, a pyrazolone derivative, an oxazole derivative, a fluorenone derivative, a hydrazone derivative, a stilbene derivative, a phthalocyanine derivative, a porphyrin derivative, a polyvinylcarbazole, a polysilylene, or a polythiophene may be mentioned.

In addition, as an electron transport material used for an electron transport layer, for example, an oxadiazole derivative, an oxazole derivative, a triazole derivative, a thiadiazole derivative, a pyrazine derivative, a triazole derivative, a triazine derivative, a perylene derivative, a quinoline derivative, a quinoxaline derivative, a fluorenone derivative, an anthrone derivative, a phenanthroline derivative, or an organic metal complex, such as a quinolinol aluminum complex, may be mentioned.

Through various research carried out by the present inventors, it was found that a device using the benzoxazolyl carbazole compound according to the present invention as a host material of the light-emitting layer had a high light-emitting efficiency and was driven by a low voltage. In a blue phosphorescent organic light-emitting device, the light-emitting peak wavelength is a short wavelength of 450 to 470 nm, and a known host material which can satisfy high lowest triplet energy as described above is limited. As a material having high lowest triplet energy, for example, a carbazole compound may be mentioned.

Incidentally, in a light-emitting layer in which high energy chemical species, such as holes, electrons, and excitons, are present at a high density, it is significantly important that a material to be used be chemically stable.

However, since having a chemically active nitrogen-hydrogen bond, carbazole itself cannot be used as a host material.

In addition, although carbazole has high hole mobility, the electron mobility thereof is low. That is, a novel compound has been desired which has both chemical stability and charge mobility of holes and electrons and at the same time which maintains the triplet energy high.

Through intensive research carried out to obtain the above desired compound, it was found that the benzoxazolyl carbazole compound according to the present invention was useful as a host material for a phosphorescent organic light-emitting device. While maintaining the triplet energy high as a carbazole compound, the compound according to the present invention can improve the chemical stability and the electron mobility.

In the benzoxazolyl carbazole compound according to the present invention, the bonding position between benzoxazole and carbazole is devised. Benzoxazole and carbazole are bonded with the nitrogen of carbazole and the carbon of the oxazole ring. Accordingly, the chemical stability is obtained. When the above two compounds are bonded at positions different from those described above, two positions each having a high chemical activity, that is, the nitrogen-hydrogen bonding position of carbazole and the 2-position of benzoxazole, which is the carbon-hydrogen bonding position, remain. In addition, benzoxazole has been used as an electron transport layer, and the electron mobility thereof is high.

That is, as described above, while having the chemical stability, the benzoxazolyl carbazole compound according to the present invention has superior hole mobility and electron mobility and can also maintain the lowest triplet energy high. Accordingly, the benzoxazolyl carbazole compound according to the present invention can provide as a host material a field in which holes and electrons are preferably recombined, energy can be efficiently transferred to a guest material, and an organic light-emitting device which has a high light-emitting efficiency and which can be driven by a low voltage can be realized.

Together with the organic compound according to the present invention, if necessary, the organic light-emitting device according to this embodiment may also use at least one of known low-molecular and high-molecular weight hole injection materials, hole transport materials, host materials, guest materials, electron injection materials, and electron transport materials.

As the organic light-emitting device according to the present invention, for example, a device in which an anode, a light-emitting layer, and a cathode are provided in this order on a substrate may be mentioned. Besides the above device, for example, a device in which an anode, a hole transport layer, an electron transport layer, and a cathode are provided in this order may also be mentioned. In addition, for example, there may also be mentioned a device in which an anode, a hole transport layer, a light-emitting layer, an electron transport layer, and a cathode are provided in this order, and a device in which an anode, a hole injection layer, a hole transport layer, a light-emitting layer, an electron transport layer, and a cathode are provided in this order. Furthermore, for example, a device in which an anode, a hole transport layer, a light-emitting layer, a hole/exciton blocking layer, an electron transport layer, and a cathode are provided in this order may also be mentioned. However, examples of these five types of multilayer organic light-emitting devices simply have very basic device structures, and the structure of an organic light-emitting device using the compound according to the present invention is not limited thereto. For example, various lamination structures may be formed in which, for example, an insulating layer is provided at the interface between an electrode and an organic compound layer, an adhesion layer or an interference layer is provided, and an electron transport layer or a hole transport layer is formed of two layers having different ionization potentials.

As the device structure in the above case, a top emission structure in which light is extracted from a substrate-side electrode or a bottom emission structure in which light is extracted from a side opposite to a substrate may be used, and in addition, a dual emission structure may also be used.

A material having a high hole mobility is preferably used as a hole injection material or a hole transport material. As a low-molecular weight and a high-molecular weight material having hole injection properties or hole transport properties, for example, there may be mentioned a triarylamine derivative, a phenylenediamine derivative, a stilbene derivative, a phthalocyanine derivative, a porphyrin derivative, a polyvinylcarbazole, a polythiophene, and other conductive polymers; however, the above material is not limited thereto.

As an electron injection material or an electron transport material, a material is selected in consideration of, for example, the balance with the hole mobility of a hole injection material or a hole transport material. As a material having electron injection properties or electron transport properties, for example, an oxadiazole derivative, an oxazole derivative, a pyrazine derivative, a triazole derivative, a triazine derivative, a quinoline derivative, a quinoxaline derivative, a phenanthroline derivative, or an organic aluminum complex may be mentioned; however, of course, the above material is not limited thereto.

As an anode material, a material having a work function as high as possible is preferably used. For example, there may be mentioned a metal element, such as gold, platinum, silver, copper, nickel, palladium, cobalt, selenium, vanadium, or tungsten; an alloy of the metal mentioned above; or a metal oxide, such as tin oxide, zinc oxide, indium oxide, indium tin oxide (ITO), or indium zinc oxide. In addition, a conductive polymer, such as a polyaniline, a polypyrrole, or a polythiophene, may also be used. These electrode materials may be used alone or in combination. In addition, the anode may have either a monolayer structure or a multilayer structure.

On the other hand, as a cathode material, a material having a low work function is preferably used. For example, there may be mentioned an alkali metal, such as lithium, an alkaline earth metal, such as calcium, or a metal element, such as aluminum, titanium, manganese, silver, lead, or chromium. In addition, an alloy containing at least two of the metal elements mentioned above may also be used. For example, magnesium-silver, aluminum-lithium, or aluminum-magnesium may be used. In addition, a metal oxide, such as indium tin oxide (ITO), may also be used. These electrode materials may be used alone or in combination. In addition, the cathode may have either a monolayer structure or a multilayer structure.

In the organic light-emitting device according to this embodiment, layers each containing the organic compound according to this embodiment and layers each containing another organic material are formed by the following method. In general, for the layer formation, for example, there may be used a vacuum deposition method, an ionization deposition method, a sputtering method, a plasma deposition method, or a known coating method, such as a spin coating method, a dipping method, a casting method, an LB method, or an ink jet method, which uses a suitable solvent for forming a solution. When the layer is formed by a vacuum deposition method, a solution coating method, or the like, for example, crystallization is not likely to occur, and excellent stability with time can be obtained. When the layer is formed by a coating method, a film may also be formed in combination with a suitable binder resin.

As the binder resin mentioned above, for example, a polyvinylcarbazole resin, a polycarbonate resin, a polyester resin, an ABS resin, an acrylic resin, a polyimide resin, a phenol resin, an epoxy resin, a silicone resin, or a urea resin may be mentioned; however, the binder resin is not limited thereto. In addition, as the binder resins mentioned above, a homopolymer or a copolymer may be used alone or in combination. Furthermore, if necessary, known additives, such as a plasticizer, an antioxidant, and an ultraviolet absorber, may also be used together with the binder resin.

### Application of Organic Light-Emitting Device

The organic light-emitting device according to the present invention may be used for a display apparatus or an illumination device. Besides the above application, the organic light-emitting device according to the present invention may also be used for an exposure light source of an electrophotographic image forming apparatus, a backlight of a liquid crystal display apparatus, and the like.

A display apparatus has the organic light-emitting devices according to this embodiment at a display portion. This display portion has a plurality of pixels. This pixel has the organic light-emitting device of this embodiment and a TFT device which is an example of a switching device for controlling light-emitting brightness, and an anode or a cathode of this organic light-emitting device is connected to a drain electrode or a source electrode of the TFT device. The display apparatus may be used as an image display device of a personal computer (PC) or the like.

The display apparatus may be an image output apparatus which has an image input portion inputting information from an area CCD, a linear CCD, a memory card, or the like and which outputs an inputted image on an display portion. In addition, the display apparatus may include an image output function which displays an image based on image information inputted from the outside as a display portion of an image forming apparatus or an ink jet printer and may also include an input function which inputs processed information for an image as an operation panel. In addition, the display apparatus may be used for a display portion of a multifunctional printer.

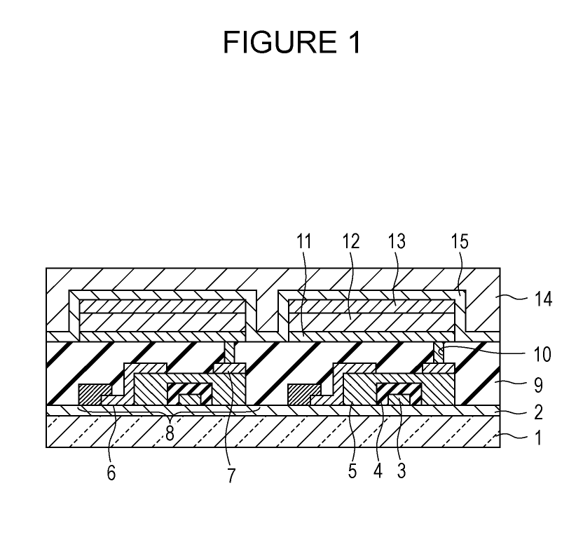

Next, a display apparatus which uses the organic light-emitting device according to this embodiment will be described with reference to FIG. 1.

FIG. 1 is a schematic cross-sectional view showing the organic light-emitting device according to this embodiment and a TFT device which is one example of a switching device connected to the above organic light-emitting device. In this FIGURE, two sets each including the organic light-emitting device and the TFT device are shown. The structure will be described in detail.

The display apparatus shown in FIG. 1 has a substrate 1 of a glass or the like, and a moisture-preventing film 2 provided thereon for protecting a TFT device or an organic compound layer. In addition, reference numeral 3 indicates a metal gate electrode. Reference numeral 4 indicates a gate insulating film, and reference numeral 5 indicates a semiconductor layer.

A TFT device 8 includes the semiconductor layer 5, a drain electrode 6, and a source electrode 7. An insulating film 9 is provided over the TFT devices 8. An anode 11 of the organic light-emitting device and the source electrode 7 are connected to each other through a contact hole 10. The structure of the display apparatus is not limited to that described above, and one of the source electrode and the drain electrode of the TFT device may be connected to one of the anode and the cathode.

Although including a plurality of organic compound layers, in this FIGURE, an organic compound layer 12 is shown as if being formed of a single layer. In order to suppress degradation of the organic light-emitting device, a first protective layer 14 and a second protective layer 15 are provided on a cathode 13.

In the display apparatus according to this embodiment, the switching device is not particularly limited, and for example, a single crystal silicon device, a metal-insulator-metal (MIM) device, or an amorphous silicon type device may be used.

### EXAMPLES

Hereinafter, the present invention will be described in detail with reference to examples. However, the present invention is not limited thereto.

### Example 1

### Synthesis of Compound H-08

### (1) Synthesis of Intermediate A-03

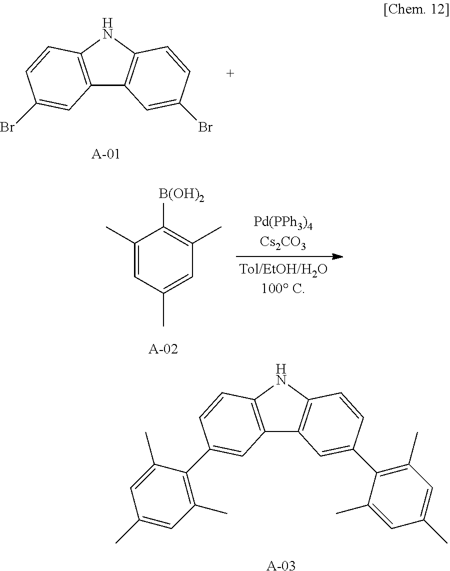

After 13.2 g (41 mmol) of the above A-01 (trade name: 3,6-Dibromocarbazole, manufactured by Wako Pure Chemical Industries, Ltd.), 20 g (122 mmol) of A-02 (trade name: 2,4,6-trimethylphenylboronic acid, manufactured by Wako Pure Chemical Industries, Ltd.), 0.938 g (0.8 mmol) of tetrakis(triphenylphosphine)palladium (Pd(PPh₃)₄), 40 g (123 mmol) of Cs₂CO₃, 150 ml of toluene, 100 ml of ethanol, and 100 ml of water were prepared and were then added in a 500 ml flask, a reaction was carried out by stirring at 100° C. for 8 hours.

After a reaction mixture was cooled to room temperature, a separated organic layer was extracted and condensed, and a residue obtained thereby was purified by chromatography on a silicagel column (EtOAc/heptane=1/4), so that 9.8 g (24 mmol) of an intermediate A-03 was obtained. The yield was 60%.

### (2) Synthesis of Exemplified Compound H-08

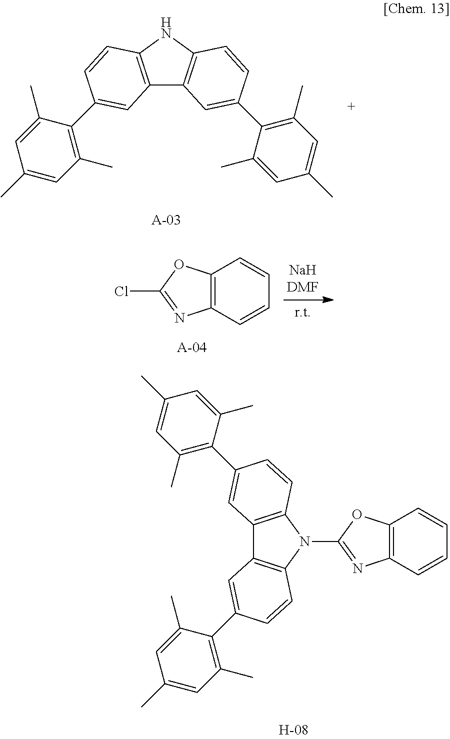

To a 200 ml flask, 2.5 g (6.2 mmol) of the above intermediate A-03, 70 ml of dimethylformamide (DMF), and 0.335 g (8.3 mmol) of NaH (60 percent by weight) were added in this order. After stirring was performed at room temperature for 20 minutes, 1.2 g (7.8 mmol) of the above A-(trade name: 2-Chlorobenzoxazole, manufactured by Tokyo Chemical Industry Co., Ltd.) was charged. After stirring was performed at room temperature all night long, the reaction was quenched by adding 50 ml of water, and washing with methanol and suction filtration were then performed. A residue thus obtained was recrystallized (toluene/EtOH=1/1). Furthermore, a recrystallized powder thus obtained was dried and sublimed (10⁻⁴ Pa, 320° C.), and 1.53 g (2.9 mmol) of the compound H-08 was obtained. The yield was 47%.

NMR (CDCl₃): δ 8.7 (2H), 87.7 (3H), 87.6 (1H), 87.3 to 7.4 (4H), 87.0 (4H), 82.3 (6H), 82.0 (12H).

MALDI-MS: 520.3

### Example 2

When the 0-0 band of phosphorescence of a toluene solution (concentration; 10⁻³ mol/l) of the compound H-08 obtained in Example 1 at 77K was measured by a spectrophotofluorometer Hitachi F-4500, the lowest triplet energy level was 419 nm.

### Example 3

### Device Evaluation

An organic light-emitting device having the structure shown in FIG. 1 was formed by the following method.

On a glass substrate, indium tin oxide (ITO) functioning as an anode was formed by a sputtering method to have a thickness of 120 nm. The ITO film thus formed was patterned so that the anode had an area of 4 mm².

Next, ultrasonic washing was sequentially performed using ultrapure water and isopropyl alcohol (IPA). Furthermore, UV/ozone washing was performed, so that a support substrate having transparency and conductivity was prepared.

Next, as a hole injection material, a known hole transport material (trade name: 4,4′,4″-Tris(carbazol-9-yl)-triphenylamine, (popular name; TCTA), manufactured by Luminescence Technology Corp. (Taiwan)) represented by the following formula (I) was deposited on the ITO electrode, so that a hole transport layer having a thickness of 50 nm was formed.

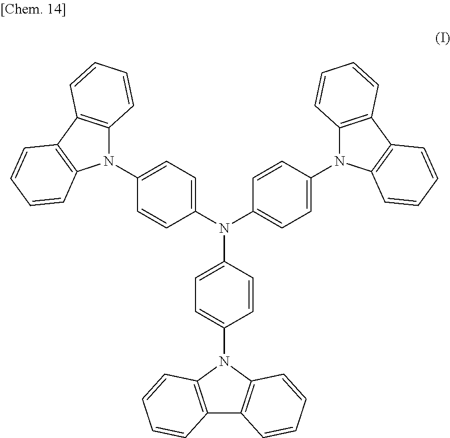 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

Next, on the hole transport layer thus formed, a phosphorescent light-emitting Ir complex represented by the following formula (II) and the benzoxazolyl carbazole compound H-08 obtained in Example 1 were codeposited at different deposition rates so that the concentration of the Ir complex was 10 percent by weight with respect to the compound H-08, thereby forming a light-emitting layer having a thickness of 40 nm. The degree of vacuum in deposition for film formation was set to 2.0×10⁻⁵ Pa.

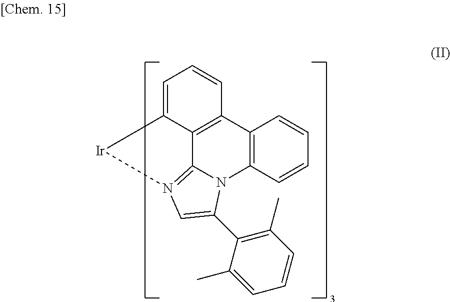

On the light-emitting layer, a compound represented by the following formula (III) was deposited, so that an electron transport layer having a thickness of 30 nm was provided. The degree of vacuum in deposition was set to 2.0×10⁻⁵ Pa, and the film-formation rate was set to 0.1 nm/sec.

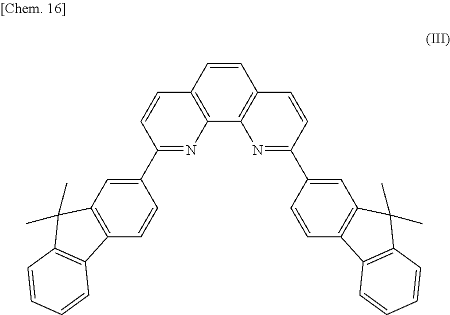

Next, lithium fluoride (LiF) was deposited to have a thickness of 0.5 nm as a cathode, and aluminum (Al) was further deposited to have a thickness of 120 nm. The degree of vacuum in deposition was set to 4.0×10⁻⁵ Pa, the film-formation rates of lithium fluoride (LiF) and aluminum (Al) were set to 0.015 nm/sec and 0.4 to 0.5 nm/sec, respectively.

The organic light-emitting device thus obtained was covered with a protective glass in a dry air atmosphere and was then sealed with an epoxy-based adhesive so as to prevent device degradation caused by moisture absorption.

When a voltage applied to the device thus obtained at a light-emitting brightness of 500 cd/m² was measured using the ITO electrode as a positive electrode and the LiF/Al electrode as a negative electrode, 9.7 V was obtained, and blue light emission having a light-emitting efficiency of 5.3 lm/W was observed.

### Comparative Example 1

A device was formed in a manner similar to that of Example 3 except that the following comparative compound R-01 disclosed in PTL 2 was used instead of the compound H-08 of Example 1, and evaluation similar to that described above was performed. The voltage applied at a light-emitting brightness of 500 cd/m² was 13.8 V, and blue light emission having a light-emitting efficiency of 3.0 lm/W was observed.

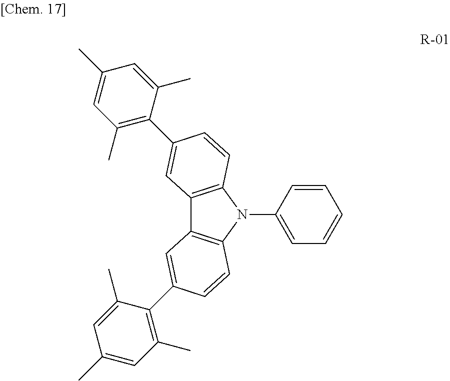 [SMILES: Cc1cc(C)c(-c2ccc3c(c2)c2cc(-c4c(C)cc(C)cc4C)ccc2n3-c2ccccc2)c(C)c1]

From the above results, it was found that when the benzoxazolyl carbazole compound according to the present invention was used as an organic light-emitting material, the light-emitting efficiency was increased, and a low voltage drive could be realized.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-075663, filed Mar. 29, 2010, which is hereby incorporated by reference herein in its entirety.

### INDUSTRIAL APPLICABILITY

The technique according to the present invention may be applied not only to a display apparatus, such as a full color display apparatus, but also to an illumination device, an apparatus using a photoelectric transducer, an electrophotographic apparatus, and the like.

1. A benzoxazolyl carbazole compound represented by the following general formula [1] 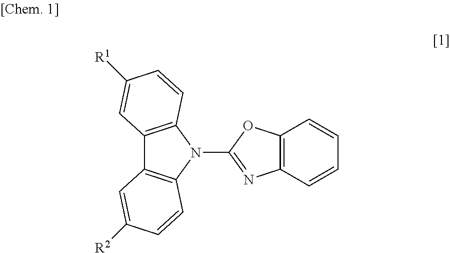
where R¹ and R² independently indicate a non-substituted phenyl group or a phenyl group substituted with at least one alkyl group having 1 to 6 carbon atoms.

2. The compound according to claim 1,
wherein the R¹ and R² each indicate one of a tolyl group, a xylyl group, and a mesityl group.

3. An organic light-emitting device comprising:
a pair of electrodes arranged to face each other; and
one or more organic layers disposed therebetween,
wherein at least one organic layer is a light-emitting layer containing the compound according to claim 1.

4. The organic light-emitting device according to claim 3,
wherein the light-emitting layer is a light-emitting layer containing a phosphorescent light-emitting Ir complex as a guest material.

5. An image display apparatus comprising:
the organic light-emitting device according to claim 3; and
a thin film transistor provided in combination therewith.

6. An image display apparatus comprising:
the organic light emitting device according to claim 4; and
a thin film transistor provided in combination therewith.

7. An image display apparatus comprising:
the organic light emitting device according to claim 3; and
switching element provided in combination therewith.

8. An image display apparatus comprising:
the organic light emitting device according to claim 4; and
switching element provided in combination therewith.
